(19) United States
(12) Patent Application Publication
(10) Pub. No.: US 2011/0245849 A1
(43) Pub. Date: Oct. 6, 2011

(54) Systems and Methods for Accommodating Anatomical Characteristics in the Treatment of Septal Defects

(76) Inventors: Ronald J. Jabba, Redwood City, CA (US); Ryan Abbott, San Jose, CA (US); Dean Carson, Mountain View, CA (US); James Nielsen, San Francisco, CA (US); W. Martin Belef, San Jose, CA (US)

(21) Appl. No.: 13/161,287

(22) Filed: Jun. 15, 2011

Related U.S. Application Data
(63) Continuation of application No. 12/113,842, filed on May 1, 2008, now abandoned.
(60) Provisional application No. 60/916,264, filed on May 4, 2007.

(51) Int. Cl. A61B 17/10

(52) U.S. Cl. 606/142

(57) ABSTRACT

Systems and methods for treating internal tissue defects, such as septal defects, with implantable devices are provided. An exemplary clip-based device includes a tubular body having at least a deflectable anchors coupled thereto. The anchors can be coupled on opposite ends of the tubular body and configured to deflect between an undeployed configuration and a deployed configuration. In the deployed configuration, each anchor extends outwardly away from the tubular body in a position configured to abut a tissue surface. The anchors are preferably configured to maintain a tissue wall therebetween and at least partially close any opening in the tissue wall. Also provided are delivery devices for delivering the implantable closure device and methods for using the various devices.

### CROSS-REFERENCE TO RELATED APPLICATIONS

This application is a continuation of U.S. patent application Ser. No. 12/113,842, filed May 1, 2008, now abandoned, which claims the benefit to U.S. Provisional Application Ser. No. 60/916,264, filed May 4, 2007, each of which is fully incorporated herein by reference.

### FIELD OF THE INVENTION

The inventions described herein relate generally to the treatment of septal defects and more particularly, to the treatment of patent foramen ovales (PFOs) while accommodating anatomical characteristics in the cardiac tissue.

### BACKGROUND OF THE INVENTION

Various defects can occur in the inter-atrial and inter-ventricular septal walls of the heart. For instance, abnormal openings in the inter-atrial septal wall can allow blood to shunt between the left and right atria. Inter-atrial defects can be generally classified as atrial septal defects (ASDs) or patent foramen ovales (PFOs). An ASD is generally defined as a direct opening in the septal wall that can allow blood to flow relatively unobstructed between the left and right atria. A PFO is generally defined as an opening existing between two flaps of septal tissue, referred to as the septum primum and the septum secundum. Between the left and right ventricles, other septal defects known as ventricular septal defects (VSDs) can exist, which are generally defined as direct openings in the ventricular septal wall that can allow blood to flow relatively unobstructed between the left and right ventricles. Another type of cardiac defect, which is generally grouped together with the aforementioned septal defects, is a patent ductus arteriosus (PDA), which is an abnormal shunt between the aorta and pulmonary artery. characteristics of the tissue surrounding the defect, which are generally not apparent to those of skill in the art. For instance, very little in published literature describes variations that can occur in the tissue during the pressure changes that occur within a typical cardiac cycle. Furthermore, devices that seek to treat many of these defects using a transcatheter, or other remote percutaneous procedure, also must take into account the geometry of the access route to the septal defect as well as variations that can occur in that geometry either between patients, or within the cardiac cycle of the patient.

Accordingly, improved systems and methods for treating septal defects, which accommodate anatomical characteristics of the surrounding tissue and vasculature, are needed.

### SUMMARY

Provided herein are systems and methods configured to treat septal defects and other internal tissue defects. These systems and methods are provided in this section by way of exemplary embodiments that should not be construed as limiting the systems and methods in any way.

In one exemplary embodiment, an implantable closure device having a clip-like configuration is provided. In another exemplary embodiment, a delivery device for delivering the implantable closure device is provided. In other exemplary embodiments, these closure and delivery devices are configured to treat septal defects while accommodating the anatomical nature, dimensions and characteristics of the defect and the surrounding anatomy.

Other systems, methods, features and advantages of the invention will be or will become apparent to one with skill in the art upon examination of the following figures and detailed description. It is intended that all such additional systems, methods, features and advantages be included within this description, be within the scope of the invention, and be protected by the accompanying claims. It is also intended that the invention is not limited to require the details of the example embodiments.

### BRIEF DESCRIPTION OF THE FIGURES

The details of the invention, both as to its structure and operation, may be gleaned in part by study of the accompanying figures, in which like reference numerals refer to like parts. The components in the figures are not necessarily to scale, emphasis instead being placed upon illustrating the principles of the invention. Moreover, all illustrations are intended to convey concepts, where relative sizes, shapes and other detailed attributes may be illustrated schematically rather than literally or precisely.

FIG. 1A is an exterior/interior view depicting an example human heart.

FIG. 1B is an enlarged side view of the septal wall depicting a PFO taken from the right atrium.

FIG. 1C is an enlarged side view of the septal wall depicting a PFO taken from the left atrium.

FIG. 1D is a cross-sectional view depicting an example PFO region taken along line 2D-2D of FIGS. 1B-C.

FIG. 2A is a side view depicting an exemplary embodiment of an implantable closure device.

FIG. 2B is a perspective view depicting an exemplary embodiment of an implantable closure device.

FIG. 2C is a top down view depicting an exemplary embodiment of an implantable closure device.

FIGS. 3A-C are perspective views depicting an exemplary embodiment of a delivery device.

FIG. 4A is a cross-sectional view of left and right atria in a heart having a PFO.

FIG. 4B is a cross-sectional view of left and right atria in a heart having a PFO with an exemplary embodiment of a delivery device therein.

FIG. 5A is a cross-sectional view depicting a septal wall.

FIG. 5B is a cross-sectional view depicting an exemplary embodiment of a delivery device engaged with a septal wall.

FIG. 5C is a perspective view depicting another exemplary embodiment of a treatment system.

FIG. 6A is a cross-sectional view depicting an exemplary embodiment of needle member after it has penetrated a septum secundum.

FIG. 6B is a cross-sectional view depicting an exemplary embodiment of needle member after it has penetrated a septum secundum and a septum primum.

FIG. 7A is a cross-sectional view depicting an exemplary embodiment of an implantable closure device implanted within a septal wall.

FIG. 7B is a graph depicting a septal thickness over one cardiac cycle.

FIG. 8 is a top view taken from the right atrium depicting exemplary embodiments of an implantable closure device implanted within a PFO tunnel.

### DETAILED DESCRIPTION

Provided herein are systems and methods for treating septal defects configured to accommodate anatomical characteristics and dimensions of the defects and the surrounding anatomy. Deformable clip-type devices for treating septal defects are described herein, along with systems for delivery of those devices as well as methods for using the same. For ease of discussion, these devices, systems and methods will be described with reference to treatment of a PFO. However, it should be understood that these devices, systems and methods can be used in treatment of any type of septal defect including ASD's, VSD's and the like, as well as PDA's, pulmonary shunts or other structural cardiac or vascular defects or non-vascular defects, and also any other tissue defect including non-septal tissue defects.

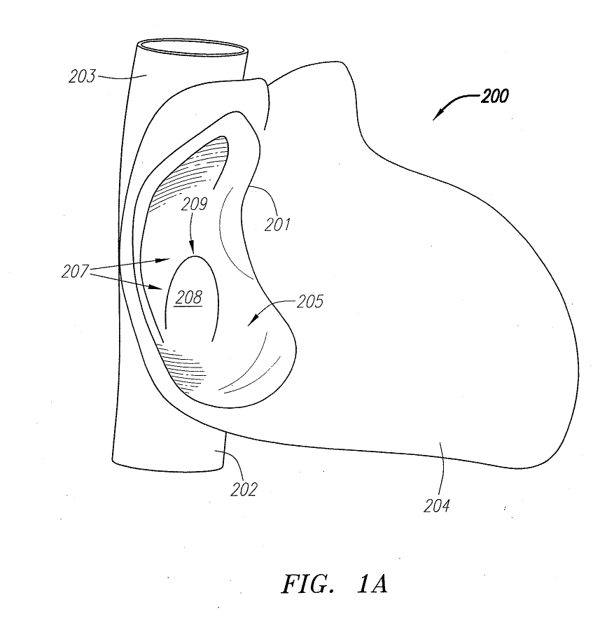

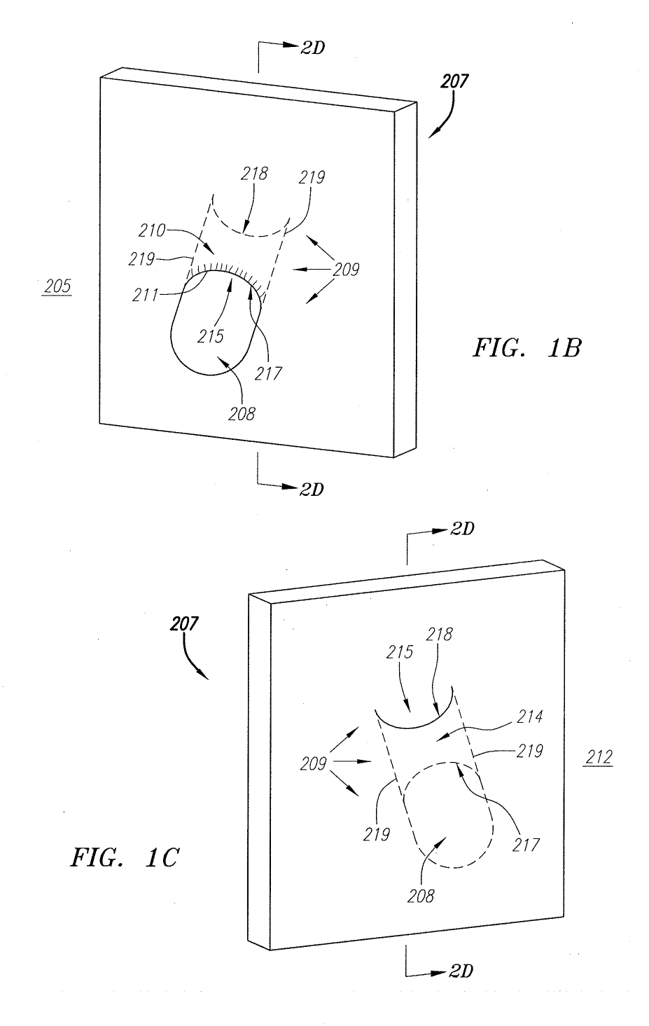

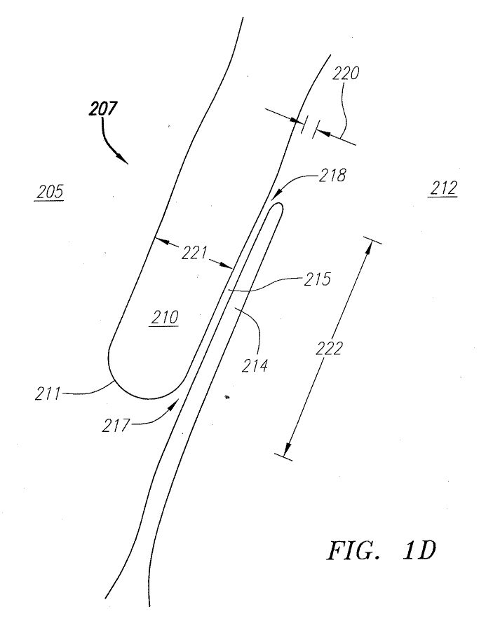

To ease the description of the many alternative embodiments of the systems and methods described herein, the anatomical structure of an example human heart having a PFO will be described in brief. FIG. 1A is an exterior/interior view depicting an example human heart 200 with a portion of the inferior vena cava (IVC) 202 and the superior vena cava (SVC) 203 connected thereto. Outer tissue surface 204 of heart 200 is shown along with the interior of right atrium 205 via cutaway portion 201. Depicted within right atrium 205 is septal wall 207, which is placed between right atrium 205 and the left atrium located on the opposite side (not shown). Also depicted is fossa ovalis 208, which is a region of septal wall 207 having tissue that is relatively thinner than the surrounding tissue. PFO region 209 is located beyond the upper portion of the fossa ovalis 208.

FIG. 1B is an enlarged view of septal wall 207 depicting PFO region 209 in more detail as viewed from right atrium 205. PFO region 209 includes septum secundum 210, which is a first flap-like portion of septal wall 207. The edge of this flap above fossa ovalis 208 is referred to as the limbus 211. FIG. 1C is also an enlarged view of septal wall 207, instead depicting septal wall 207 as viewed from left atrium 212. Here, PFO region 209 is seen to include septum primum 214, which is a second flap-like portion of septal wall 207. Septum primum 214 and septum secundum 210 partially overlap each other and define a tunnel-like opening 215 between sidewalls 219 (indicated as dashed lines in FIGS. 1B-C) that can allow blood to shunt between right atrium 205 and left atrium 212 and is commonly referred to as a PFO.

FIG. 1D is a cross-sectional view depicting an example PFO region 209 taken along line 2D-2D of FIGS. 1B-C. Here, it can be seen that septum secundum 210 is thicker than septum primum 214. Typically, the blood pressure within left atrium 212 is higher than that within right atrium 205 and tunnel 215 remains sealed. However, under some circumstances, conditions can occur when the blood pressure within right atrium 205 becomes higher than the blood pressure within left atrium 212 and blood shunts from right atrium 205 to left atrium 212 (e.g., a valsava condition). Because most typical shunts occur in this manner and for purposes of facilitating the discussion herein, region 217 in FIG. 1D will be referred to as PFO entrance 217, and region 218 will be referred to as PFO exit 218.

Many different variations of PFO's can occur. For instance, thickness 220 of septum primum 214, thickness 221 of septum secundum 210, overlap distance 222 and the flexibility and distensibility of both septum primum 214 and septum secundum 210 can all vary. In FIGS. 1B-C, PFO entrance 217 and PFO exit 218 are depicted as being relatively the same size with the width of tunnel 215, or the distance between sidewalls 219, remaining relatively constant. However, in some cases PFO entrance 217 can be larger than PFO exit 218, resulting in an tunnel 215 that converges as blood passes through. Conversely, PFO entrance 217 can be smaller than PFO exit 218, resulting in an opening that diverges as blood passes through. Furthermore, multiple PFO exits 218 can be present, with one or more individual tunnels 215 therebetween. Also, in FIGS. 1B-D, both septum primum 214 and septum secundum 210 are depicted as relatively planar tissue flaps, but in some cases one or both of septum primum 214 and septum secundum 210 can have folded, non-planar, or highly irregular shapes.

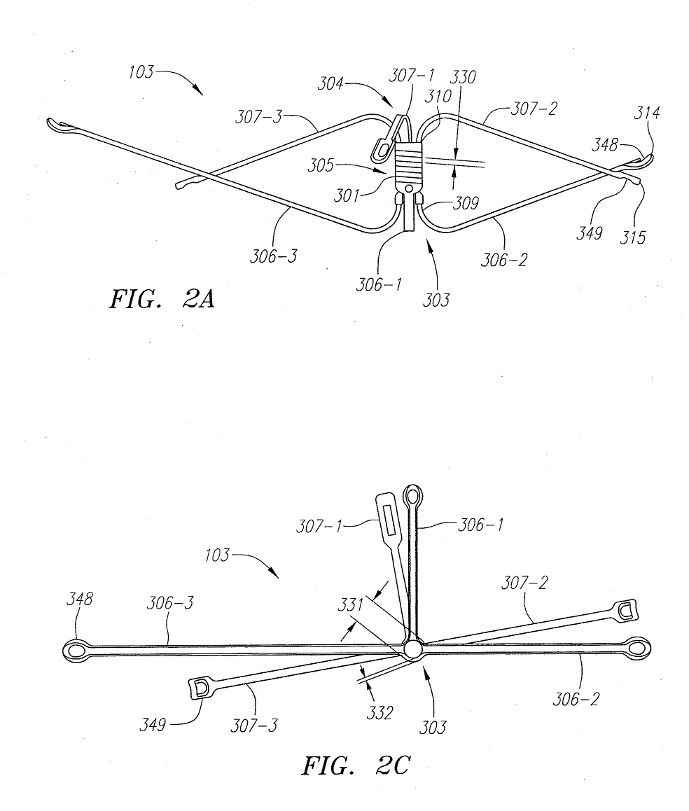

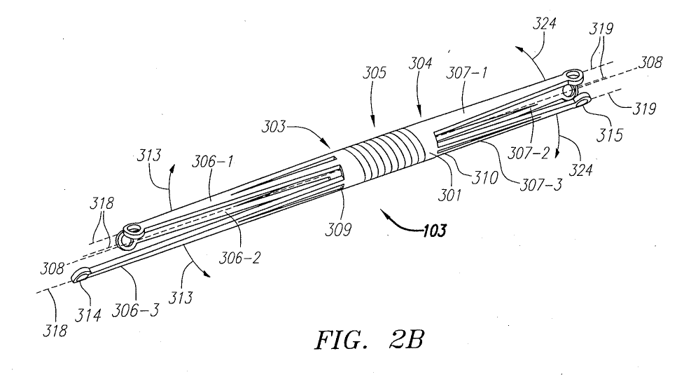

FIGS. 2A-C are various views depicting an exemplary embodiment of an implantable device 103 configured to facilitate partial or entire closure of a PFO. In this embodiment, device 103 has a clip-like configuration similar to that described in U.S. patent application Ser. Nos. 11/295,338 entitled “Clip-Based Systems and Methods for Treating Septal Defects,” filed Dec. 5, 2005, 11/427,572 entitled “Systems And Methods For Treating Septal Defects,” filed Jun. 29, 2006, and 11/744,784 entitled “Systems And Methods For Treating Septal Defects,” filed May 4, 2007, each of which are fully incorporated by reference herein. To facilitate the description herein, device 103 will be referred to herein as clip 103, although it is not limited to such.

FIG. 2A is a side view depicting clip 103 in an at-rest configuration. Clip 103 is preferably biased to deflect to this at-rest configuration from the undeployed, or housed, configuration depicted in the side view of FIG. 2B. This housed configuration allows clip 103 to be carried within the inner lumen of a needle or other tissue piercing structure as will be described below. Clip 103, after deployment from within the needle through the septum primum and secundum, transitions towards the at-rest state and preferably compresses the opposing flaps together to close the PFO tunnel existing therebetween. FIG. 2C is a top down view depicting clip 103 in a typical deployed configuration similar to that configuration that can occur once implanted. It should be noted that while clip 103 can transition all of the way towards the at-rest configuration, clip 103 is preferably configured such that the presence of the septal tissue restricts the full transition, allowing clip 103 to maintain a compressive force on the septal tissue.

In this embodiment, clip 103 has a body 301 and includes a distal portion 303, a proximal portion 304 and a tubular central portion 305, which is preferably a bendable, compressible and/or expandable portion, and is configured as a coil in this embodiment. Clip 103 includes three left atrial (LA) members 306-1, 306-2 and 306-3 and three right atrial (RA) members 307-1, 307-2 and 307-3. Each of anchors 306 and 307 are deflectable (i.e., bendable, shiftable, twistable or turnable) from the housed configuration to the at-rest configuration. In this embodiment, members 306-307 have two primary functions, to act as anchors for clip 103 and to act to compress the septal tissue. For purposes of facilitating the description herein, members 306-307 will be referred to as anchors 306-307.

Here, LA anchors 306 are coupled to distal end 309 of distal end portion 303 of clip 103 and RA anchors 307 are coupled to proximal end 310 of proximal end portion 304. LA anchor 306-3 has a length relatively longer than the other LA anchors 306-1 and 306-2, and will be discussed in more detail below.

LA anchors 306 and RA anchors 307 have end tips 314 and 315, respectively, that are preferably atraumatic. Here, tips 314 and 315 are annular to be atraumatic to tissue, and include inner apertures 348 and 349, respectively. Inner apertures 348 and 349 allow tissue to mechanically anchor to implant 103 in order to reduce chronic abrasion and potential tissue perforation risks. Although not shown, the atraumatic characteristics of end tips 314 and 315 can be improved by deflecting them away from any adjacent tissue surface. Also, radiopaque markers (e.g., tantalum) can be placed within apertures 348 and 349 (or anywhere on clip 103) to increase the visibility of clip 103 in x-ray imaging. Radiopaque markers can be also be placed within the tubular body of clip 103 itself, to increase the visibility of clip 103 and prevent residual shunting through clip 103.

FIG. 2B depicts each anchor 306 and 307 oriented generally along main axis 308 of body 301. Arrows 313 and 324 indicate the direction in which each LA and RA member 306 and 307, respectively, is biased to deflect. In the undeployed configuration, the entire body 301 of clip 103, including anchors 306 and 307, has a generally elongate shape, in this case being describable as rod-like or cylindrical.

As shown in FIG. 2B, each LA and RA anchor 306 and 307 can be described as having a longitudinal axis 318 and 319, respectively. LA longitudinal axis 318 extends from a base portion 309 of each LA anchor 306 to end tip 314. Likewise, RA longitudinal axis 310 extends from a base portion 321 of each RA anchor 307 to end tip 315. In the undeployed configuration, these longitudinal axes 318 and 319 are oriented generally along main axis 308, although not necessarily parallel with main axis 308. In the deployed configuration, each longitudinal axis 318 and 319 is offset from main axis 308 by a relatively greater amount than in the undeployed configuration. Viewed differently, longitudinal axes 318 and 319 can be described as being relatively less parallel to main axis 308 in the deployed configuration than in the undeployed configuration. It should be noted that LA and RA anchors 306 and 307 are not required to be straight in order to have a longitudinal axis 318 and 319, respectively.

Referring now to FIG. 2C, it can be seen that the LA anchors 306 are radially offset from the RA anchors 307. This allows for the various anchors to achieve at greater amount of deflection in the at-rest configuration of FIG. 2A. Offsetting the anchors 306-307 with respect to each other prevents them from contacting each other in the at-rest configuration and allows them to deflect past each other. This, in turn, allows for the application of greater compressive force when clip 103 is deployed in the septal tissue.

As mentioned above, central portion 305 of body 301 is preferably configured to be bendable, expandable and/or compressible to facilitate closure of the PFO tunnel. In this embodiment, central portion 305 is configured to be an elastic, spring-like portion of body 301. Central portion 305 is preferably biased towards a fully compressed state to effectuate the maximum closure force onto septal wall 207 and the PFO tunnel. Central portion 305 can expand to accommodate varying thickness of septal wall 207, i.e., in the event that septal wall 207 is thicker than the length of body 301 between LA anchors 306 and RA anchors 307.

Clip 103 is preferably fabricated from a superelastic material such as NITINOL and the like or an elastic material such as stainless steel and the like, so as to provide the desired biased deflections or shape altering characteristics. Any shape memory characteristics of the material (e.g., NITINOL) can also be incorporated into the functional operation of clip 103. For instance, in one exemplary embodiment, body 301 is composed of NITINOL and heat treated in the deployed configuration so as to instill that shape. A typical heat treatment procedure can occur for 1-20 minutes in a temperature range of 500-550° C. based on factors such as the heating device and the clip material, although clip 103 is not limited to heat treatment in only that range of time and temperature. The process steps and conditions for heat treating NITINOL to instill a desired shape is well known to those of ordinary skill in the art. After heat treatment, members 306 and 307 become biased towards the deployed configuration such that members 306 and 307 will remain deformable yet will resist any deflection or movement away from that configuration. Members 306 and 307 can then be deflected into the undeployed configuration so that clip 103 can be loaded into delivery device 104 (e.g., needle 120, sheath 123, etc.). Therefore, upon exposure of clip 103 from within delivery device 104, members 306 and 307 will begin to return to the heat-treated, deployed configuration.

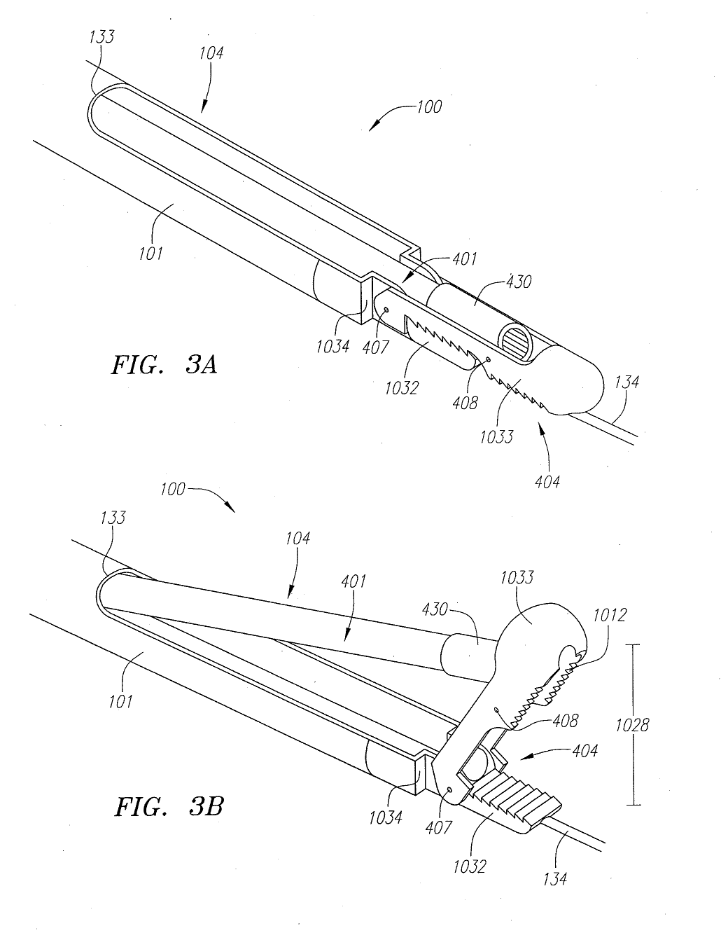

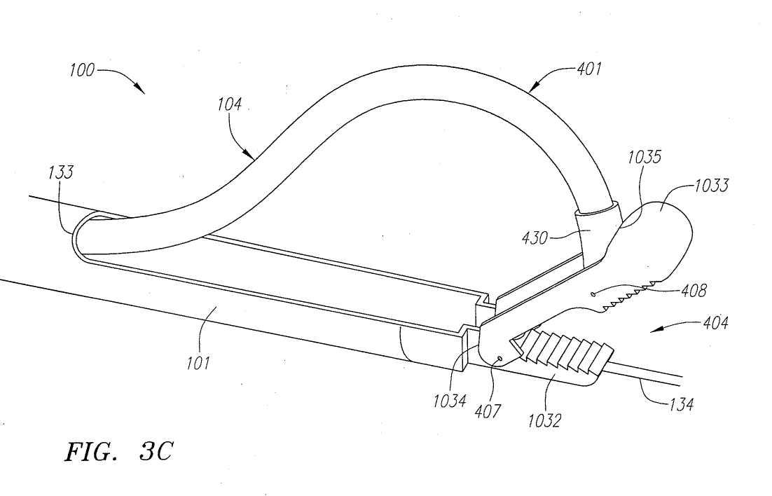

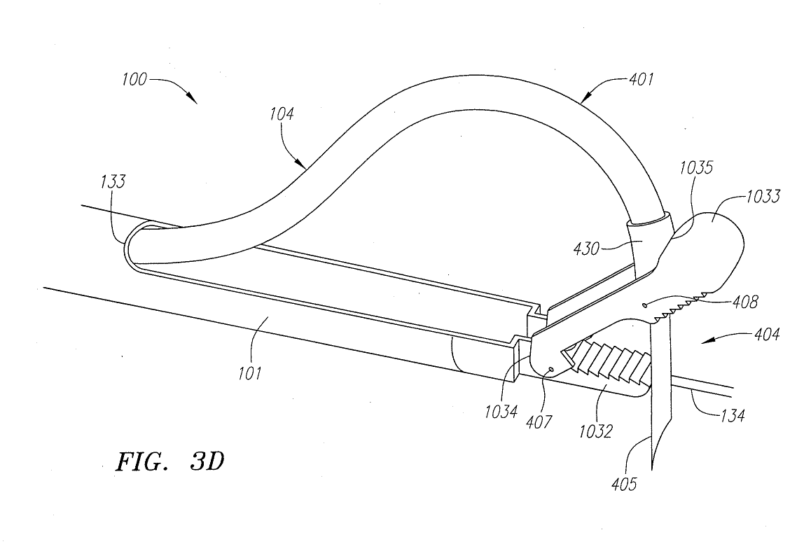

Clip 103 is preferably configured for use with treatment system 100. Treatment system 100 preferably includes a delivery device 104, which is depicted in FIGS. 3A-C. Delivery device 104 preferably utilizes an off-axis delivery technique to implant clip 103. Such an off-axis delivery technique is described in detail in co-pending U.S. patent application Ser. No. 11/363,961 entitled “Methods and Devices for Delivery of Prosthetic Heart Valves and Other Prosthetics,” which is fully incorporated herein by reference. The use of a similar treatment systems 100, also employing off-axis techniques, are described in detail in co-pending U.S. patent application Ser. Nos. 11/175,814, filed Jul. 5, 2005 and entitled “Systems and Methods for Treating Septal Defects,” and 11/218,794, filed Sep. 1, 2005 and entitled “Suture-based Systems and Methods for Treating Septal Defects,” both of which are fully incorporated by reference herein. Although these applications are directed mainly to the delivery of coil-like and suture-like devices, respectively, many of the delivery methods and systems that are described are equally applicable to clip 103. Similar treatment systems are also described in the above incorporated application Ser. Nos. 11/295,338 entitled “Clip-Based Systems and Methods for Treating Septal Defects,” filed Dec. 5, 2005, 11/427,572 entitled “Systems And Methods For Treating Septal Defects,” filed Jun. 29, 2006, and 11/744,784 entitled “Systems And Methods For Treating Septal Defects,” filed May 4, 2007.

FIG. 3A is a prospective view depicting an exemplary embodiment of delivery device 104 configured for off-axis delivery. Delivery device 104 can include body member 101 having one or more lumens located herein. Within one lumen of body member 101 can be an axially slidable off-axis (OA) delivery member 401. On the distal end of body member 101 is a tissue engagement device 404 which can include a lower portion 1032 and an upper portion 1033 each being pivotably coupled together.

The distal end of OA delivery member 401 is pivotably coupled with upper portion 1033 at a position proximal to the distal end of portion 1033. Portions 1032 and 1033 each include one or more abutments or teeth 1012, which can give portions 1032 and 1033 a forcep (or grasper)-like function. For ease of discussion herein, portions 1032 and 1033 will be referred to as lower jaw 1032 and upper jaw 1033, although they are not limited to such. Distal tip 430 of OA delivery member 401 also includes teeth 1012 at its distal end.

FIG. 3A depicts delivery device 104 in an undeployed state suitable for advancement through the vasculature of the patient. This advancement can occur along the length of guidewire 134, which is preferably routed into position beforehand. Once in proximity with limbus 211 of septal wall 207, the user preferably exerts a proximal force on delivery member 401 to cause tissue engagement device 404 to open. In this embodiment, delivery member 401 pulls upper jaw 1033 proximally causing it to rotate, or pivot, with respect to lower jaw 1032 by way of pivot 407. Distal tip 430 of delivery member 401 in turn rotates with respect to upper jaw 1033 by way of pivot 408. A proximal stop 1034 is positioned to stop the proximal movement of upper jaw 1033 when retracted proximally.

FIG. 3B is a perspective view depicting this exemplary embodiment after tissue engagement device 404 has been opened. As can be seen here, upper jaw 1033 has been raised and stopped at a height generally shown as height 1020. Height 1020 will be referred to herein as the “clamp distance” of device 404. In this position delivery device 104 can be advanced distally against the limbus such that the limbus enters the gap created between jaws 1032 and 1033 of tissue engagement device 404. Once the limbus is positioned within device 404 as desired, a distal force is preferably exerted by the user on OA delivery member 401 causing tissue engagement device 404 to close and compress the limbus between jaws 1032 and 1033. Delivery device 104 now preferably exerts force sufficient on the limbus to engage, or couple, device 104 with the secundum tissue.

Continued distal advancement of OA delivery member 401 (while body member 101 is held stationary) causes delivery member 401 to deflect upwards and outwards from body member 101 into the deployed, curved stated depicted in the perspective view of FIG. 3C. It should be noted that none of the tissue of the patient is depicted here for purposes of clarity. Such depictions are made in the above-incorporated application, U.S. patent application Ser. No. 11/363,961.

FIG. 3C depicts a needle member 405 after it has been advanced from within OA delivery member 401. Needle member 405 is preferably configured to pierce septum the secundum and primum to create a transeptal puncture through both layers of tissue to provide access to the left atrium. As described in the incorporated U.S. patent application Ser. Nos. 11/363,961, 11/295,338, 11/427,572, and 11/744,784, a pusher member (not shown) can be used to advance clip 103 from within needle 405 and deliver clip 103 into a position suitable for the closure of PFO.

Delivery device 104 is preferably configured such that when placed in the configuration for deployment of clip 103, needle 405 is placed at a desired angle with respect to the septal tissue. In this embodiment, the proper orientation of needle 405 is accomplished by a distal stop 1035 that abuts distal tip 430 of OA delivery member 401. Advancement of delivery member 401 in the distal direction will cause distal tip 430 to abut stop 1035 and cease movement of OA delivery member 401. From this position needle 405 can be advanced into septal tissue at the desired angle, which in this embodiment is approximately 90 degrees from the main axis of body member 101. The desired angle can also be controlled by the distance OA delivery member 401 is advanced with respect to body member 101. The size of the deflected arc of OA delivery member 401 is controlled in part by the location of proximal lumen opening 133, which can be a skive in body member 101, as depicted in this embodiment.

As will be described in more detail below, treatment of a PFO preferably includes inserting treatment system 100 into the vasculature of a patient and advancing body member 101 through the vasculature to the inferior vena cava (e.g., over a guidewire), from which access to the right atrium can be obtained. Once properly positioned within the right atrium, delivery device 104 can be used to deliver one or more clips 103 to the PFO region, preferably by inserting each clip 103 through septum secundum 210 and primum 214 such that it lies transverse to the PFO tunnel and exerts a force that at least partially closes the PFO tunnel. Thus, the use of clip-based devices, systems and methods for treating PFO's allows direct closure of the PFO tunnel, as opposed to occlusive-type devices that merely block the PFO entrance and exit without directly closing the tunnel.

Treatment of a PFO by transeptal placement of an implantable closure device requires the piercing of the septal tissue at an angle generally transverse to the plane of the septal wall (e.g., the plane of the primum, the secundum and the adjacent tissue). Use of a delivery device with the capability to orient the implant to travel along a path transverse to a main longitudinal axis of the device is generally referred to herein as “off-axis delivery.”

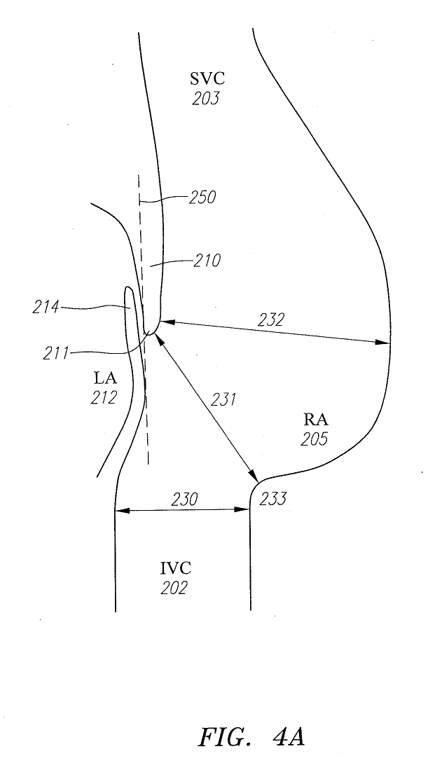

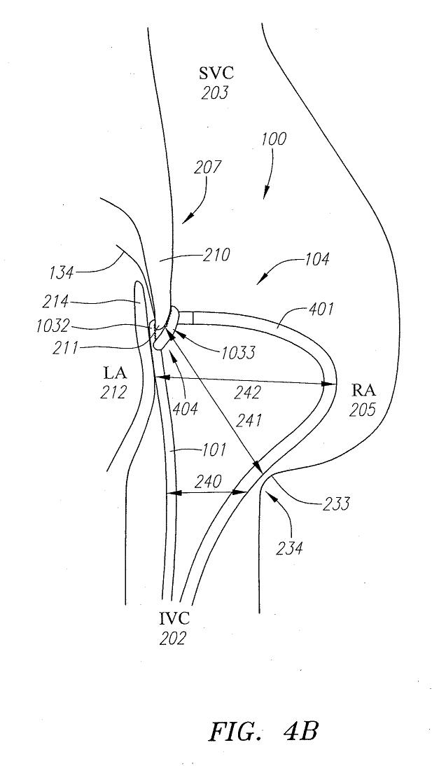

When approaching the septal wall from either the right or left atrium, various tissue anatomy can constrain the available workspace in which off-axis delivery can be achieved. FIG. 4A is a view of right atrium 205 (similar to one that could be obtained using external imaging such as echocardiography with a bicaval view) that depicts various anatomical distances 230-232 that can constrain off-axis delivery (with respect to septal wall plane 250) when accomplished by way of a right atrial approach. Shown here is right atrium 205 with IVC 202 and SVC 203 adjacent thereto. Tissue junction 233 is the interface between the annulus of IVC 202 and right atrial chamber 205.

Here, distance 230 is the diameter of the annulus of IVC 202. Distance 231 is the distance between the limbus 211 of septum secundum 210 and the interface between IVC 202 and right atrial chamber 205. Distance 232 is the distance between septum secundum 210 generally adjacent to limbus 211 and the opposite right atrial far wall. Each of these distances 230-232 can constrain an off-axis delivery device. The constraints will depend on the actual configuration of that device, which will vary between designs and between applications. These constraints can cause bending, kinking or other distortion in OA delivery member 401. If the constraints are severe, deployment of delivery device 104 can be prevented altogether.

These distances 231-232 can additionally vary throughout a cardiac cycle. Table 1 quantifies these distances for an exemplary segment of the population.

TABLE 1

| | Smallest Point in | | | Largest Point in | | |
|---|---|---|---|---|---|---|
| | Cardiac Cycle | | | Cardiac Cycle | | |
| Distance | 230 | 231 | 232 | 230 | 231 | 232 |
| Average | 24 | 34 | 41 | 24 | 39 | 46 |
| (mm) | | | | | | |
| Standard | 7 | 5 | 7 | 7 | 6 | 7 |
| Deviation | | | | | | |
| (mm) | | | | | | |

Table 1 provides the values for these distances at their relative smallest points in a cardiac cycle as compared to their relative largest points in a cardiac cycle. The average values for both the smallest point and largest points are provided for an exemplary segment of the population. Also provided is the degree of variance, shown as one standard deviation, among this segment of the population.

As can be seen, distance 231, between the limbus 211 and tissue junction 233 (located at the interface between the annulus of IVC 202 and right atrial chamber 205) can vary on an average of 5 millimeters (mm) during a cardiac cycle (between 34 and 39 mm for an average member of the population). Likewise, distance 232, between secundum 210 and the opposite right atrial wall 205, can also vary on an average of 5 mm during a cardiac cycle (between 41 and 46 mm for an average member of the population). Distance 230, the diameter of the annulus of IVC 202, remains relatively constant during a cardiac cycle (24 mm). Any device used to achieve off-axis orientation within IVC 202 and/or or right atrium 205 is subject to these anatomical constraints. Similar constraints exist within left atrium 212 that would need to be considered in developing an off-axis device for operation within left atrium 212.

FIG. 4B depicts an exemplary embodiment of system 100 deployed within right atrium 205. Here, system 100 has been routed through IVC 202 into right atrium 205 and is thus constrained by the anatomical dimensions 230, 231 and 232. OA delivery member 401 is preferably configured to deploy without coming into substantial contact with one or both sides of the annulus of IVC 202, the tissue junction 233 and the right atrial chamber wall. For instance, if OA delivery member 401 is in substantial contact with junction 233 then the force exhibited by junction 233 in direction 234 can cause OA delivery member 401 to bend or kink or otherwise inhibit the advancement of needle or pusher member within. Furthermore, OA delivery member 401 preferably is configured to avoid any application of force by junction 233 while the position of that junction is varying during the cardiac cycle. For instance, as shown in Table 1, distance 231 between limbus 211 and junction 233 will vary within a cardiac cycle and thus can create temporary kinks, bends or other distortions in OA delivery member 401.

OA delivery member 401 is also constrained by the size of the right atrial chamber 205, which is indicated generally by distance 232. OA delivery member 401 is preferably configured to deploy to a distance less than the minimum length of distance 232 at its smallest point in the cardiac cycle. Contact of delivery member 401 with the right atrial wall can cause bending, kinking or other distortion in, or movement of delivery member 401 that can inhibit the deployment of clip 103. While in this embodiment the effects of distances 231 and 232 result in distortion in OA delivery member 401's preferred deployment profile, the actual effects of the tissue anatomy on a given delivery device will vary based on the configuration of that device. Thus, one of skill in the art will readily recognize that different devices will be affected by the anatomy in different ways.

Preferably, the dimensions of device 104 in its deployed and expanded state are less than the anatomical constraints 230-232. Because the anatomical constraints vary among members of the population, delivery device 104 is preferably configured to accommodate a desired target percentage of the population. Multiple different delivery devices 104 can be provided to a medical professional, each being configured to deploy within anatomies of varying degrees of size. Alternatively, one delivery device 104 can be configured to deploy within a large subset of the population.

For instance, in one exemplary embodiment delivery device 104 is configured to deploy within the anatomy of an average member of the population. In this instance, distance 240, as shown in FIG. 4B, is the width of device 104 at the annulus of IVC 202, and is preferably less than 24 mm. Distance 241, which coincides with the distance 231 between limbus 211 and tissue junction 233, is preferably less than 34 mm. Distance 242, which coincides with distance 232 between septal wall 207 and opposite the right atrial wall, is preferably less than 41 mm. Another exemplary embodiment device 104 is configured at dimensions one standard deviation smaller than the average. In this embodiment, distance 240 is 17 mm, distance 241 is 29 mm, and distance 242 is 34 mm.

However, it should be understood that the device can be configured with any desired dimensions so long as the device is not significantly adversely impacted by the anatomical constraints. Furthermore, anti-kinking catheter designs, such as those described in U.S. patent application Ser. No. 11/744,784, entitled “Systems and Methods for Treating Septal Defects” filed May 4, 2007, can be used to increase the robustness of device 104 and allow the dimensions of device 104 to be further reduced to avoid any of the aforementioned constraints.

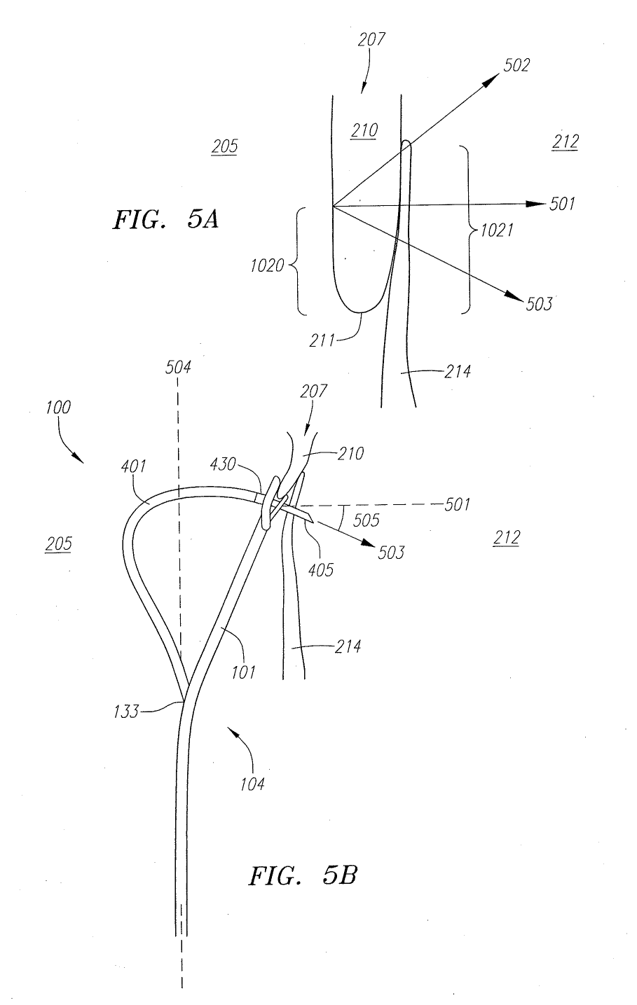

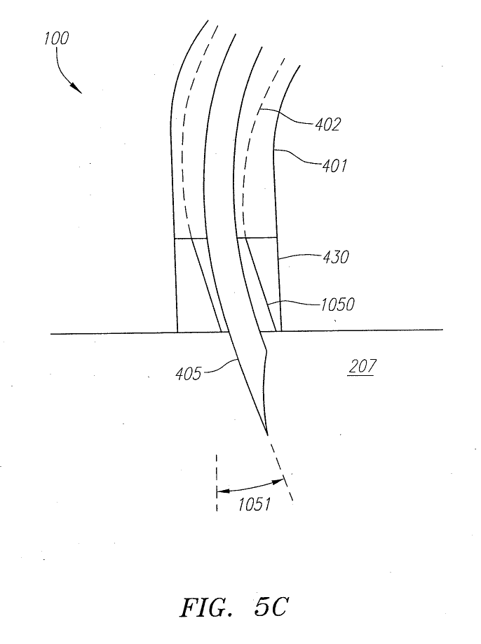

FIG. 5A is a cross-sectional view depicting septal wall 207. Here, various trajectories 501-503 are depicted through septal wall 207. These trajectories can be the trajectory of needle 405 or any other tissue piercing structure. The distance from limbus 211 at which needle 405 preferably enters secundum 210 can be affected by the clamp distance 1028. This distance is preferably configured to allow adequate amounts of septal tissue to be engaged by clip 103 after implantation. If clamp distance 1028 is too small, the tissue piercing structure may not adequately engage secundum 210 or may slip off of secundum 210 altogether. Preferably, clamp distance 1028 should be greater than the puncture distance, i.e., the distance from the edge of the limbus to the point on the outer surface to the secundum where the needle penetrates, although the clamp distance can be equal to or less than the puncture distance in other embodiments. The puncture distance is preferably in a range of 3-7 mm. However, different devices 104 can be capable of operating at different minimal puncture distances. In one exemplary embodiment, clamp distance 1028 is 3-4 mm greater than the puncture distance.

In cases where the puncture distance is relatively long, it is possible for the tissue piercing structure to miss primum 214 altogether, or at least not puncture an adequate amount of septal tissue in primum 214 (e.g., creating the risk that the primum tissue will tear loose). For instance, if the puncture distance is greater than PFO tunnel length 1021, then a generally perpendicular trajectory for the tissue piercing structure, such as that indicated by trajectory 501, would miss primum 214 altogether. This is generally undesirable for septal procedures where transeptal puncture of both secundum 210 and primum 214 is desired.

Conversely, even if the puncture distance is kept to a minimum, an excessively short tunnel length 1021 can still result in failure to pierce an adequate amount of primum 214. FIG. 5A depicts an instance where the puncture distance 1020 is less than tunnel length 1021. Here, when the tissue piercing structure takes a trajectory that is less than 90 degrees, as depicted by trajectory 502, inadequate primum capture can result, either by piercing too little primum tissue or missing primum altogether. It is desirable therefore to configure delivery device 104 to cause the needle trajectory to be greater than 90 degrees as exemplified by trajectory 503. With trajectory 503, variations in puncture distance 1020 and/or tunnel length 1021 become less likely to result in inadequate tissue capture. Any amount of downward trajectory greater than 90 degrees will increase the likelihood to achieve adequate capture of primum 214.

FIG. 5B depicts an exemplary embodiment of delivery device 104 engaged with septum secundum 210. In this embodiment, delivery device 104 is configured to achieve a generally downward sloping needle trajectory. Here, the downward sloping trajectory is approximately 30 degrees greater than the 90 degree trajectory depicted by reference line 501. In this embodiment, to achieve this downward sloping trajectory, delivery device 104 is configured to deflect from its main axis 504. The advancement of OA delivery member 401 into the off-axis position depicted here causes force to be exerted on body member 101 both at the distal tip 430 of member 401 and at the proximal lumen opening 133. These forces act in conjunction to cause the deflection of the distal portion of delivery device 104 away from main axis 504 by an amount which equals angle 505 when axis 504 is generally perpendicular to the normal trajectory 501 as shown in FIG. 5B.

As can be seen in FIG. 5B, as a result of the action of device 104, limbus 211 and secundum 210 deflect outwards into right atrium 205 away from its natural disposition. In this embodiment, the trajectory of a needle (not shown) through secundum 210 is generally perpendicular to the plane of secundum 210. However, because secundum 210 is deflected outwards the net effect is to create a downward sloping needle trajectory 503 which adequately captures primum 214. In another embodiment, the position of stop 1035 (not shown) can be adjusted to achieve the desired trajectory. Device 104 can be configured to achieve any incremental degree of downward trajectory greater than about 90 degrees (e.g., 95°, 105°, 120°).

FIG. 5C is a perspective view depicting another embodiment of system 100 where needle member 405 is configured to enter septal wall 207 at an angle (device 404 is omitted for clarity). In this embodiment, the angle at which needle 405 enters septal wall 207 can be adjusted in any direction desired. Lumen 1050 of distal tip 430 is positioned through distal tip 430 at an angle 1051. This in turn causes needle 405 to deploy from distal tip 430 at the same or similar angle 1051. By adjusting angle 1051, needle 405 can be made to enter septal wall 207 at an angle with respect to OA delivery member 401. Although deflection can occur in any direction (e.g., to the left, right, or distally or proximally), in this embodiment deflection is shown to occur distally. Deflection in a proximal direction, while risking inadequate capture of the primum as described above, can be used to facilitate to passage of needle 405 through septal wall 207 without contacting lower portion 1032 (not shown).

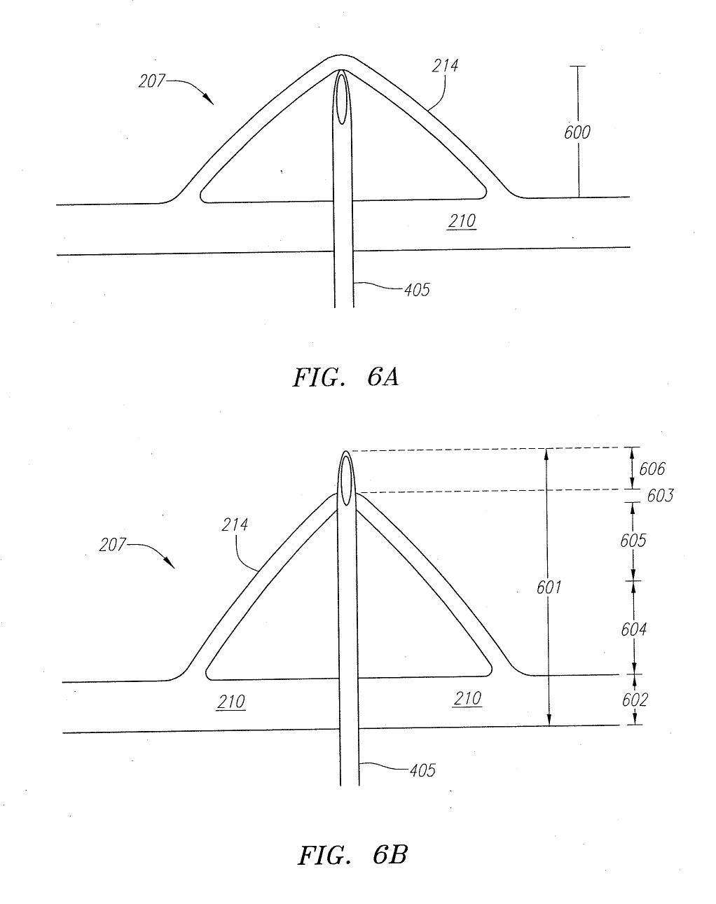

FIG. 6A is a cross-sectional view depicting an exemplary embodiment of needle member 405 after it has penetrated septum secundum 210 but prior to penetration of septum primum 214. Here it can be seen that needle member 405 has advanced a distance 600 past septum secundum 210 yet has not pierced septum primum 214. Two of the multiple factors that contribute to this are referred to as primum tenting and primum excursion. Primum excursion is the loose displacement of the primum tissue as well as the motion of the primum tissue during the cardiac cycle which can be caused by either or both of normal and abnormal factors. The primum tissue does not necessarily remain disposed adjacent to the secundum 210 at all times during the cardiac cycle. In fact, the primum tissue can be a folded, loosely disposed flap of tissue capable of variable movement with respect to secundum 210.

Primum tenting refers to the characteristics of the primum tissue in that it is distensible and stretchable. Contact with the tissue piercing structure does not necessarily result in immediate piercing of primum 214, and can instead force primum 214 to travel away from secundum 210 until the primum is distended to such an extent that further motion of the tissue piercing structure results in the actual piercing.

When implementing device 104 in a configuration suitable for transeptal puncture, a minimum distal translation of needle 405 is desired to ensure repeatable piercing of primum 214 without significant manual intervention. Often in transeptal procedures, visibility to the administering medical professional is limited and it is thus desirable to configure device 104 to achieve primum piercing on a regular and repeatable basis.

FIG. 6B is a cross-sectional view depicting needle 405 after piercing primum 214. Needle 405 is shown after traveling a minimum travel distance 601. Minimum travel distance 601 preferably results in primum puncture in a significant portion (e.g. greater than half) of the general population having PFOs. Distance 601 can include multiple, varying numbers of factors, depending on the configuration of device 104. Preferably, distance 601 includes at least distance components 602-606.

Distance 602 in FIG. 6B is defined as the thickness of secundum 210. This thickness is variable based on differences between patients as well as any effect delivery device 104 has on the thickness of secundum 210. For instance, in the embodiments of device 104 described with respect to FIGS. 3A-C, tissue engagement device 404 compresses secundum 210 such that it is not the same thickness as in its natural state. While the compressed thickness of secundum 210 will vary based on the configuration of engagement device 404 as well as the force supplied the secundum 210, generally this compressed thickness is approximately 2.5 mm for average members of the population.

Distance 603 is defined as the thickness of primum 210 which is approximately a minimum of 1 mm for average members of the population. Distances 604 and 605 relate to the amount of primum excursion and primum tenting that occurs, respectively. Generally the amount of primum excursion 604 for a majority of the population is approximately 5.8 mm. Primum tenting will generally vary based on the size of the tunnel and the individual's tissue characteristics. For instance, a 14 mm wide PFO tunnel having an 5.8 mm excursion when tented is approximately 2.5 mm. Distance 606 reflects the desire for a suitable amount of needle 405 to travel through primum 214 and is preferably half of the length of the opening in the distal end of needle 405 when viewed from the perspective shown here. This distance will vary based on the size and shape of needle 405 as well as the beveled angle (if any) that is present on the distal end of needle 405. Other tissue piercing structures will require different amounts of extra travel 606 based on the actual implementation of the tissue piercing structure. Embodiments that do not have a beveled distal surface, for example, can exclude the extra travel distance 606 altogether. The preferable minimal value of distance 601 is 14.3 mm and delivery device 104 can be configured to achieve a repeatable needle travel of at least 14.3 mm.

Other factors can also be incorporated into a minimal repeatable needle travel 601. Delivery device 104 as described with respect to FIGS. 3A-C, can be subject to a deflection of OA delivery member 401 that occurs to the left side or the right side of the device. This will generally result in needle 405 traveling at an angle not absolutely perpendicular to septal wall 207 but tilted to the left or right of the PFO tunnel by a slight amount. This can result in a disparity between the actual translation of needle 405 along the length of device 104 and the absolute travel of needle 405 through septal wall 207. In a preferred embodiment, this distance is approximately 1 mm, making distance 601 approximately 15.3 mm.

Different delivery devices 104 also are subject to different manufacturing tolerances as will be recognized by one of skill in the art. These manufacturing tolerances may also create disparity in the travel of needle 405. Also, tolerances can be introduced as a result of the route taken through the patient's vasculature. Accordingly, an additional tolerance is preferably added to provide adequate translation of needle 405 through septal wall 207. In a preferred embodiment, this tolerance is approximately 2.5 mm. Thus, in certain embodiments it is desirable to achieve a minimal travel 601 greater than 14.3 mm. In one embodiment, the minimum needle travel 601 is 18 mm+/−2 mm. It should be noted any combination of these distances and tolerances can be considered in determining the minimum travel 601. Preferably, the absolute needle travel does not exceed 35-36 mm and more preferably, the absolute needle travel is less than 30 mm.

Needle travel 601 is preferably achieved on a repeatable basis such that the user is not required to manually gauge the amount of travel either by referencing the amount of travel on the proximal end of device 104 or by referencing an image of the heart itself. Any configuration of proximal controller can be used. Some exemplary embodiments of proximal controllers are described in the above-incorporated U.S. patent application Ser. Nos. 11/427,572, and 11/744,784. These applications describe proximal controllers for use in a PFO treatment procedure. Each of these embodiments can be configured to achieve a minimal actual needle travel of the desired amount greater than or equal to distance 601.

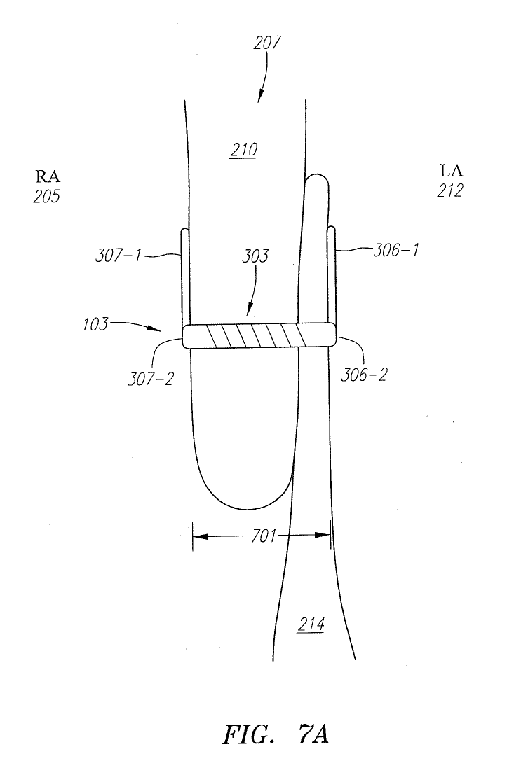

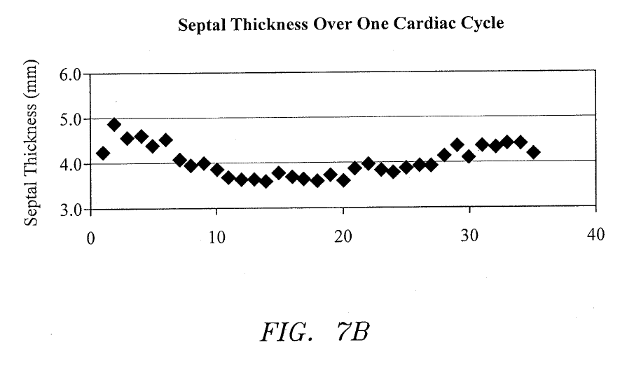

FIG. 7A is a cross-sectional view depicting an embodiment clip 103 implanted within septal wall 207. As mentioned, the coiled center section 303 can provide a compressive force to secundum 210 and primum 214 that aids in closing the PFO tunnel. If thickness 701, which is the thickness of septal wall 207 at the position of implantation of clip 103, remains constant over time, clip 103 can remain in a fairly static position and is only minimally susceptible to fatigue. However, the septal tissue thickness 701 can vary significantly during a typical cardiac cycle.

FIG. 7B is a graph depicting a septal thickness 701 over one cardiac cycle. Here it can be seen that the septal thickness can vary, on average, by 25 percent (plus-or-minus 15 percent) in the course of a cardiac cycle. This can result in significant repetitious expansion and compression of the coiled portion of clip 103 while implanted within the patient. Accordingly, clip 103 is preferably configured to accommodate this cyclic variation in thickness 701 and also do so without succumbing to material fatigue failure.

To adequately accommodate the cyclic variation in thickness of septal wall 207, the embodiment of clip 103 described with respect to FIGS. 2A-C is configured with a fatigue-resistant central section 303. Referring back to FIGS. 2A-C, this central section 303 can have a tubular shape with a six coil section, each coil having a length 330 of approximately 0.006 inch. The width 331 of coiled section 303 is approximately 0.033 inch and the wall thickness 332 of tubular section 303 is approximately 0.005 inch. Clip 103 is preferably composed of nitinol, for example, approximately 55.8% Nickel and 44.2% Titanium (with trace amounts of other materials, having a austenitic finish temperature of approximately 5 degrees Celsius. Clip 103 can also be doped with Chrome. Clip 103 is preferably configured to withstand at least 50 million cardiac cycles without a significant number of fatigue-induced failures. While the variation and thickness of septal tissue 207 can be quite significant during a cardiac cycle, due to tissue remodeling, this variation can decrease over time. For instance, after a period of several weeks, the amount of thickness variation may become negligible.

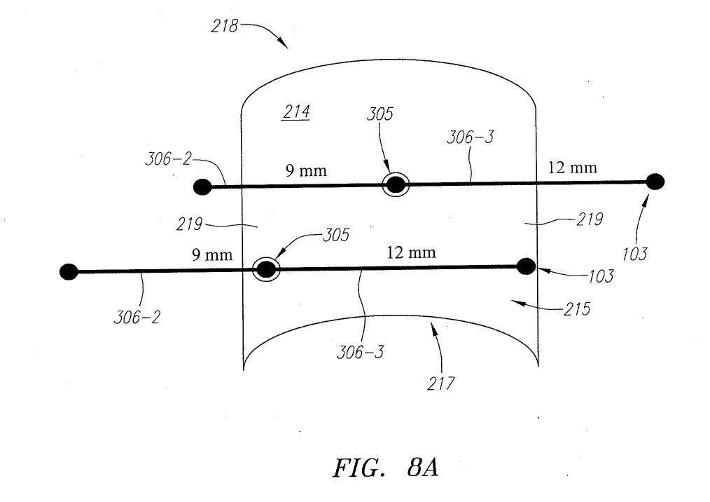

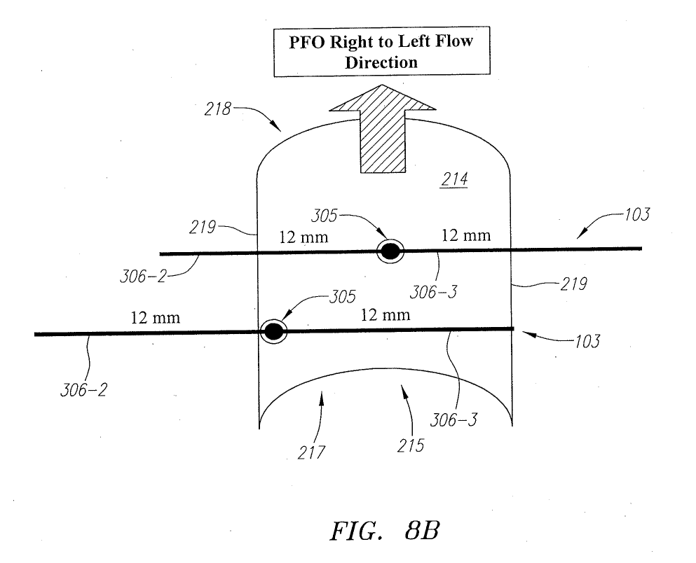

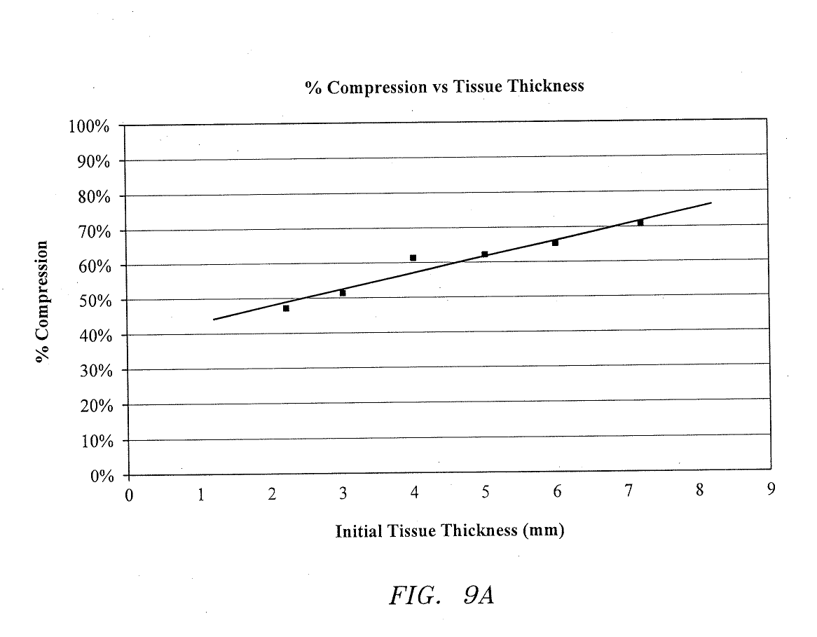

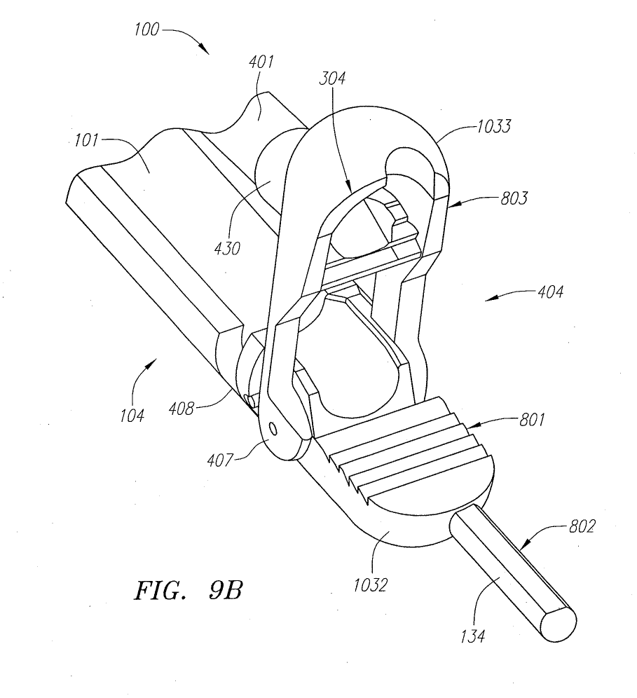

The PFO tunnel, when viewed from the right atrium, can be converging, diverging or straight and typically bends to the right. Because of this, transeptal punctures can tend to occur on the left side of the tunnel in the absence of techniques or devices that achieve a predetermined puncture position with respect to the left and right walls of the tunnel. The width of the tunnel in about 90 percent of the population having PFOs is approximately 13 mm. FIGS. 8A-B are top views taken from the right atrium (similar to FIG. 1B) depicting exemplary embodiments of clip 103 implanted within PFO tunnel 215 in two separate positions (secundum 210, LA anchor 306-3 and RA anchors 307 are not shown). The upper position is centrally disposed in PFO tunnel 215 while the lower position is implanted on the left side of the tunnel. In the embodiments of FIG. 8A, clip 103 is configured with a relatively longer left atrial anchor 306-3, which is configured to extend across the majority of the PFO tunnel in the instance that the implant is implanted in a non-central left-oriented position. Here, the PFO tunnel has a width of approximately 15 mm.

In the embodiments of FIG. 8A, LA anchor 306-2 on the left side has a length of approximately 9 mm while LA anchor 306-3 on the right side has a longer length of approximately 12 mm, while the central portion 305 has a width of approximately 1 mm. If clip 103 is implanted in a centrally disposed position, the length of LA anchors 306-2 and 306-3 is sufficient to provide full coverage across the tunnel, as shown by the upper example.

If clip 103 is implanted to the left of tunnel 215, LA anchor 306-3 is sufficiently long to maintain adequate coverage. If clip 103 is implanted while a guidewire is disposed within PFO tunnel 215, the guidewire is preferably positioned to the left of clip 103 and forces clip 103 to be implanted just adjacent to the left hand side wall of tunnel 215. This offset (approximately 2 mm) will be enough to provide that LA anchor 306-3 extends the entire way across PFO tunnel 215. Full coverage of primum 214 reduces the likelihood that a residual shunt will remain through PFO tunnel 215 after implantation. It should be noted that LA anchors 306 can be configured with any desired length, although a relatively longer LA anchor 306-3 is preferable.

In the embodiments of FIG. 8B, LA anchor 306-2 on the left side has a length of approximately 12 mm while LA anchor 306-3 on the right side also has a length of approximately 12 mm, while the central portion 305 has a width of approximately 1 mm. Regardless of where clip 103 is implanted along PFO tunnel 215, the length of LA anchors 306-2 and 306-3 is sufficient to provide full coverage across tunnel 215, as shown here.

The devices and methods herein may be used in any part of the body, in order to treat a variety of disease states. Of particular interest are applications within hollow organs including but not limited to the heart and blood vessels (arterial and venous), lungs and air passageways, digestive organs (esophagus, stomach, intestines, biliary tree, etc.). The devices and methods will also find use within the genitourinary tract in such areas as the bladder, urethra, ureters, and other areas.

Other locations in which and around which the subject devices and methods find use include the liver, spleen, pancreas and kidney. Any thoracic, abdominal, pelvic, or intravascular location falls within the scope of this description.

The devices and methods may also be used in any region of the body in which it is desirable to appose tissues. This may be useful for causing apposition of the skin or its layers (dermis, epidermis, etc), fascia, muscle, peritoneum, and the like. For example, the subject devices may be used after laparoscopic and/or thoracoscopic procedures to close trocar defects, thus minimizing the likelihood of subsequent hernias. Alternatively, devices that can be used to tighten or lock sutures may find use in various laparoscopic or thoracoscopic procedures where knot tying is required, such as bariatric procedures (gastric bypass and the like) and Nissen fundoplication. The subject devices and methods may also be used to close vascular access sites (either percutaneous, or cut-down). These examples are not meant to be limiting.

The devices and methods can also be used to apply various patch-like or non-patchlike implants (including but not limited to Dacron, Marlex, surgical meshes, and other synthetic and non-synthetic materials) to desired locations. For example, the subject devices may be used to apply mesh to facilitate closure of hernias during open, minimally invasive, laparoscopic, and preperitoneal surgical hernia repairs.

It should be noted that various embodiments are described herein with reference to one or more numerical values. These numerical value(s) are intended as examples only and in no way should be construed as limiting the subject matter recited in any claim, absent express recitation of a numerical value in that claim.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure.

What is claimed is:

1. A medical apparatus, comprising:
a delivery device configured for advancement through an inferior vena cava of a patient and into the right atrium, the delivery device comprising:
a flexible elongate body member having a distal portion with a longitudinal axis; and
an elongate delivery member coupled with the body member, the elongate delivery member having a distal portion configured to deflect away from the distal portion of the body member to an orientation where a distal end of the elongate delivery member is in a position transverse to the longitudinal axis of the body member, the delivery member being configured to avoid substantial contact with a tissue junction between an annulus of the inferior vena cava (IVC) and the right atrial wall.

2. The apparatus of claim 1, wherein the delivery device is configured to engage a limbus of the septum secundum.

3. The apparatus of claim 2, wherein the delivery device is configured to deploy within anatomy where the distance between the limbus and the tissue junction is at least 34 millimeters (mm).

4. The apparatus of claim 2, wherein the delivery device is configured to deploy within anatomy where the distance between the limbus and the tissue junction is at least 29 millimeters (mm).

5. The apparatus of claim 1, wherein the delivery device is configured to deploy within anatomy where the distance between the limbus and the tissue junction is at least 34 millimeters (mm).

6. The apparatus of claim 1, wherein the delivery device is configured to deploy within anatomy where the distance between the limbus and the tissue junction is at least 29 millimeters (mm).

7. The apparatus of claim 1, wherein the delivery device is configured to engage a limbus of the septum secundum and configured for advancement through the annulus of the IVC having a diameter of at least 24 mm.

8. The apparatus of claim 1, wherein the delivery device is configured to engage a limbus of the septum secundum and configured for advancement through the annulus of the IVC having a diameter of at least 17 mm.

9. The apparatus of claim 1, wherein the delivery device is configured for advancement through the annulus of the IVC having a diameter of at least 24 mm.

10. The apparatus of claim 1, wherein the delivery device is configured for advancement through the annulus of the IVC having a diameter of at least 17 mm.

11. The apparatus of claim 1, wherein the delivery device is configured to engage a limbus of the septum secundum and wherein the elongate delivery member is configured to deflect without substantial contact with the right atrial wall opposite the septum secundum, where the distance between the right atrial wall and the septum secundum is at least 41 mm.

12. The apparatus of claim 1, wherein the delivery device is configured to engage a limbus of the septum secundum and wherein the elongate delivery member is configured to deflect without substantial contact with the right atrial wall opposite the septum secundum, where the distance between the right atrial wall and the septum secundum is at least 34 mm.

13. The apparatus of claim 1, wherein the elongate delivery member is configured to deflect without substantial contact with the right atrial wall opposite the septum secundum, where the distance between the right atrial wall and the septum secundum is at least 41 mm.

14. The apparatus of claim 1, wherein the elongate delivery member is configured to deflect without substantial contact with the right atrial wall opposite the septum secundum, where the distance between the right atrial wall and the septum secundum is at least 34 mm.

15. An implantable medical device for treatment of a patent foramen ovale (PFO), comprising:
a body configured for trans-septal implantation through a septal wall having a PFO, the body comprising:
a first member configured to engage the septum secundum;
a second member configured to engage the septum primum; and
a compressible and expandable portion coupled with both the first and second members;
wherein the body is configured to maintain at least partial closure of a natural PFO tunnel when the thickness of the septal wall is variable over a cardiac cycle.

16. The implantable medical device of claim 15, wherein the body is configured to maintain at least partial closure when the thickness of the septal wall varies in a range of 10-40%.

17. The implantable medical device of claim 16, wherein the body is configured to maintain at least partial closure over repeated cardiac cycles.

18. The implantable medical device of claim 17, wherein the first member is configured to engage an exposed surface of the septum secundum and the second member is configured to engage an exposed surface of the septum primum.

19. The implantable medical device of claim 18, wherein the compressible and expandable portion has a first end and a second end, the first member being coupled to the first end and the second member being coupled to the second end.

20. The implantable medical device of claim 15, wherein the body is configured to accommodate at least 50 million cardiac cycles.

21. The implantable medical device of claim 15, wherein the body is configured to maintain at least partial closure when the thickness of the septal wall varies at least 25% over a cardiac cycle.

22. The implantable medical device of claim 21, wherein the body is configured to accommodate 50 million cardiac cycles.

23. The implantable medical device of claim 15, wherein the body is configured to maintain at least partial closure when the thickness of the septum secundum varies at least 25% over a cardiac cycle.

24. A system for creating a puncture in a septal wall, comprising:
a delivery device configured for movement within the vasculature of a patient, the delivery device comprising a needle member configured to puncture a septum primum and a septum secundum; and
a proximal controller coupled with the delivery device, the controller configured to control advancement of the needle member from a distal end of the delivery device by an amount of at least 14.3 millimeters.

25. The system of claim 24, wherein the proximal controller is configured to control advancement of the needle member from a distal end of the delivery device by an amount in the range of 14.3 millimeters to 30 millimeters.

26. The system of claim 24, wherein the proximal controller is configured to control advancement of the needle member from a distal end of the delivery device by an amount in the range of 16 millimeters to 20 millimeters.

27. The system of claim 24, wherein the proximal controller is configured to control advancement of the needle member from a distal end of the delivery device by an amount of approximately 18 millimeters.

28. The system of claim 24, wherein the proximal controller is configured to achieve repeatable advancement of the needle over a constant distance.

29. A system for creating a puncture in a septal wall, comprising:
a delivery device configured for movement within the vasculature of a patient, the delivery device comprising a needle member configured to puncture a septum primum and a septum secundum at an angle inclined inferior to a normal to a plane generally defined by the septum primum and septum secundum.

30. The system of claim 29, wherein the delivery device comprises a lumen configured to slidably receive the needle member, a distal end of the lumen being configured to guide the needle along a trajectory offset from the longitudinal axis of a distal end of the delivery device.

31. The system of claim 29, wherein the delivery device is configured to adjust between a first configuration for advancement through the vasculature of the patient and a second configuration for performing a trans-septal puncture, wherein the delivery device is configured to orient the needle member at the angle inclined inferior to the normal to the plane generally defined by the septum primum and septum secundum when the delivery device is in the second configuration.

32. The system of claim 29, wherein the angle is at least 5 degrees.

33. The system of claim 29, wherein the angle is at least 15 degrees.

34. The system of claim 29, wherein the angle is at least 30 degrees.

35. An implantable closure device for closure of a patent foramen ovale (PFO), comprising:
an elongate body having a first end and a second end;
a first anchor member coupled to the first end of the elongate body;
a second anchor member coupled to the first end of the elongate body; and
a third anchor member coupled to the second end of the elongate body, wherein the second and third anchor members are configured for deployment within the left atrium of a patient and configured to extend towards the left and right sides of a PFO tunnel across an area to the left and right of a PFO tunnel having a width of at least 22 millimeters.

36. The device of claim 35, wherein the second anchor member is an elongate member having a length of at least nine millimeters and the third anchor member is an elongate member having a length of at least twelve millimeters and the elongate body has a width of at least one millimeter.

37. The device of claim 35, wherein the second anchor member is an elongate member having a length of nine millimeters and the third anchor member is an elongate member having a length of twelve millimeters and the elongate body has a width of one millimeter.

38. The device of claim 35, wherein the area has a width of at least 25 millimeters.

39. The device of claim 38, wherein the second anchor member is an elongate member having a length of at least twelve millimeters and the third anchor member is an elongate member having a length of at least twelve millimeters and the elongate body has a width of at least one millimeter.

40. The device of claim 38, wherein the second anchor member is an elongate member having a length of twelve millimeters and the third anchor member is an elongate member having a length of twelve millimeters and the elongate body has a width of one millimeter.

41. The device of claim 38, wherein the second and third anchor members are configured to contact septal tissue along substantially their entire length.

42. The device of claim 38, wherein the second and third anchor members are configured to contact septal tissue over all of the portion of the area adjacent the elongate body.

43. The device of claim 35, wherein the second and third anchor members are configured to contact septal tissue over all of the portion of the area adjacent the elongate body.

44. The device of claim 35, wherein the second and third anchor members are configured to contact septal tissue along substantially their entire length.

45. A medical method, comprising:
positioning a closable tissue engagement device in a right atrium of the heart of a human patient, the heart having a patent foramen ovale (PFO) with a septum secundum, wherein the tissue engagement device is located on a distal portion of an elongate device positioned within the vasculature of the patient and the amount of closure of the tissue engagement device is controllable by a proximal portion positioned external to the patient; and
closing the tissue engagement device on a portion of the secundum until the thickness of that portion is reduced to an amount greater than 15 percent of the initial thickness of that portion prior to closure.

46. The method of claim 45, wherein closing the tissue engagement device further comprises closing the tissue engagement device on the portion of the secundum until the thickness of the portion is reduced to an amount between 15 percent and 80 percent of the initial thickness of that portion prior to closure.

47. The method of claim 45, wherein closing the tissue engagement device further comprises closing the tissue engagement device on the portion of the secundum until the thickness of the portion is reduced to an amount greater than 45 percent of the initial thickness of that portion prior to closure.

48. The method of claim 45, wherein closing the tissue engagement device further comprises closing the tissue engagement device on the portion of the secundum until the thickness of the portion is reduced to an amount between 45 percent and 80 percent of the initial thickness of that portion prior to closure.

49. The method of claim 45, further comprising implanting a PFO closure device configured to at least partially close the patent foramen ovale.

50. The method of claim 49, wherein implanting the PFO closure device further comprises implanting the PFO closure device such that it resides entirely through at least one of the septum primum and the septum secundum, after closing the tissue engagement device.

51. The method of claim 49, wherein implanting the PFO closure device further comprises implanting the PFO closure device such that it resides entirely through the septum secundum, after closing the tissue engagement device.

52. The method of claim 49, wherein implanting the PFO closure device further comprises implanting the PFO closure device such that it resides entirely through the septum secundum and the septum primum, after closing the tissue engagement device.

53. The method of claim 45, further comprising piercing entirely through the septum secundum after closing the tissue engagement device.

54. The method of claim 53, further comprising implanting a closure device in the piercing in the septum secundum.

55. The method of claim 54, wherein the tissue engagement device is maintained in the closed position until after the septum secundum has been pierced.

56. The method of claim 54, wherein the tissue engagement device is maintained in the closed position until after the PFO closure device has been partially deployed.

57. The method of claim 45, wherein the tissue engagement device comprises two opposing members pivotably coupled together.

58. The method of claim 45, further comprising the proximal controller, configured with a predetermined discrete state at which the tissue engagement device is closed such that the thickness of the portion of the secundum is reduced to an amount between 15 percent and 80 percent of the initial thickness prior to closure.

59. The method of claim 45, further comprising the proximal controller, configured with a predetermined discrete state at which the tissue engagement device is closed such that the thickness of the portion of the secundum is reduced to an amount between 45 percent and 80 percent of the initial thickness prior to closure.

60. The method of claim 45, further comprising the proximal controller, configured with a predetermined discrete state at which the tissue engagement device is closed such that the thickness of the portion of the secundum is reduced to an amount greater than 15 percent of the initial thickness prior to closure.

61. The method of claim 45, further comprising the proximal controller, configured with a predetermined discrete state at which the tissue engagement device is closed such that the thickness of the portion of the secundum is reduced to an amount greater than 45 percent of the initial thickness prior to closure.

62. The method of claim 45, wherein closing the tissue engagement device further comprises closing the tissue engagement device on the portion of the secundum until the thickness of the portion is reduced to an amount between 55 percent and 69 percent of the initial thickness of that portion prior to closure.
